(19) United States
(12) Patent Application Publication
(10) Pub. No.: US 2005/0153320 A1
(43) Pub. Date: Jul. 14, 2005

(54) Single base extension

(76) Inventors: James N. Herron, Salt Lake City, UT (US); Samuel Tolley, (US)

Correspondence Address: TRASK BRITT, P.O. BOX 2550, SALT LAKE CITY, UT, 84110, US

(21) Appl. No.: 10/984,629

(22) Filed: Nov. 8, 2004

Related U.S. Application Data
(60) Provisional application No. 60/517,660, filed on Nov. 6, 2003.

(51) Int. Cl. C12Q 1/68; C12P 19/34

(52) U.S. Cl. 435/6; 435/91.2

(57) ABSTRACT

Genetic polymorphisms can change protein structure and function, altering dispositions to diseases and conditions. A single nucleotide polymorphism is the smallest genetic mutation and the most difficult to detect. However, single nucleotide polymorphisms also make up 90% of known genetic mutations, thus identifying such polymorphisms is essential. Single base extension uses the affinity of one base for its complementary base to detect polymorphisms, including single nucleotide polymorphisms. Planar waveguides are used as the platform for single base extension enabling rapid, real time detection of genetic polymorphisms. Detection limits in the picomolar range can be obtained. Signals from the non-matched DNA bases are in the range of the blank signal. Detection times of 5 minutes are reported.

### CROSS-REFERENCE TO RELATED APPLICATIONS

This application claims the benefit under 35 U.S.C. § 119 of U.S. Provisional Patent Application No. 60/517,660, filed Nov. 6, 2003, which is hereby incorporated by reference.

### GOVERNMENT FUNDING

This project received federal funding in the form of NIH grant R01 HL32132. The U.S. government may have rights in this invention.

### TECHNICAL FIELD

The invention relates generally to biotechnology and diagnostics, and more specifically to an enzyme-catalyzed single base extension reaction used to detect single nucleotide polymorphisms with planar waveguide fluorescence biosensor technology.

### BACKGROUND

Long QT syndrome (LQTS) is a congenital genetic disorder affecting ion channels in the muscle cells of the heart (1, 2). In most cases, the cause of LQTS is a single nucleotide polymorphism (SNP) in one of six genes that encode for the ion channels. Such a polymorphism can result in a single amino acid change within an ion channel that selectively conducts ions across the cell membrane. In some cases, the structure and function of the channel will be altered, resulting in a slightly decreased ion flow under normal operation. The reduced ion flow gives rise to the major symptom of LQTS—a prolonged re-polarization time after contraction, observed as an increased time between the Q and the T waveforms in an electrocardiogram. Serious complications may arise when an affected individual experiences emotional stress or exercises vigorously. The increased requirements for cardiac ion flow under such conditions can produce a dramatic conformational change in the affected ion channels that inhibits nearly all ion flux. This inhibition can lead to torsade de pointes, ventricular fibrillation and death (2). Upon the onset of these symptoms, it is often too late for treatment. Thus, it is essential to diagnose the disorder before symptoms occur.

The two procedures used in present-day medical practice for detecting LQTS have drawbacks. The first procedure is an analysis of the patient's electrocardiogram for detection of a prolonged QT interval (2). This procedure is confounded by the natural variability in electrocardiograms: approximately 33% of carriers have normal QT intervals. The second procedure involves screening the patient's DNA for possible single nucleotide polymorphisms in the six genes that encode for the ion channels. Although fundamentally more accurate, genetic screening procedures currently used have several shortcomings. One such shortcoming occurs when the selected ion channel genes are amplified in order get enough DNA to perform the assay. Not only does this amplification take an excessive amount of time, but it can also introduce new mutations in the copies of the target DNA. The greater the amplification required, the greater the risk of replication infidelity.

The current methodology for detecting polymorphisms, is referred to as “single-strand conformational polymorphism” (3). This methodology is based on single-stranded DNA, which hybridizes intramolecularly to form secondary structures such as hairpin loops. The intermolecular hybrids are then separated via electrophoresis, depending upon size and shape. The slight conformational differences between the wild type DNA and the polymorphic type DNA are enough to cause a difference in the single strand shape, and thus in their retention on a gel. The single-chain conformational polymorphism assay was initially developed for the research environment because of its ability to identify unknown polymorphisms. Unfortunately, it is not well suited for genetic screening of patients in clinical environments because of the low throughput capabilities. An alternative is an affinity assay or hybridization assay that uses the binding energetics of base pair formation as the mechanism of selectivity rather than subtle changes in conformation within a single-stranded DNA molecule. Detection of single nucleotide polymorphisms (SNP) using a conventional hybridization assay on planar waveguides requires stringent control of reaction conditions (counter ion concentration and reaction temperature) to ensure hybridization fidelity [18, 19]. For example, the temperature of the hybridization reaction has to be strictly controlled (e.g., hybridization at 50° C. must be controlled to within 0.2° C.) for a given set of counterion (Na⁺, K⁺, Mg²⁺) concentrations [18]. Such tight temperature control is challenging in clinical laboratory environments, let alone point-of-care settings.

Although genetic screening is clearly the preferred method of LQTS detection, the problems of time impracticality, replication infidelity, control of reaction conditions and accurate resolution of single nucleotide polymorphisms hamper the advancement of such screening.

### SUMMARY OF THE INVENTION

The invention includes an enzyme-catalyzed single base extension reaction used to detect single nucleotide polymorphisms with planar waveguide fluorescence biosensor technology. Reactions may be performed at a fixed temperature between about 30° C. to about 80° C., preferably between about 40° C. and 50° C. Reaction times are typically ten minutes or less.

Single base extension on planar waveguides produce a rapid, real time detection of genetic polymorphisms. The temperature required is only 40° C., which is easily attainable in a lab or point of care setting.

Using the waveguide technology, a wash-less assay can be preformed. This increases the speed and throughput of the assay. This makes for a better choice as a point of care diagnostic system.

The detection limit calculated in the Examples herein is 30 pM. This limit is better than Single strand conformational polymorphism assays, but comparable to the simple hybridization assays done previously with the same instrument. The cost of each assay goes down as the required time for DNA amplification and the amount of reactants decreases.

The specificity of the reaction for the complementary base is excellent because it uses the binding energetics of base pair formation as the mechanism of selectivity rather than subtle changes in conformation within a single-stranded DNA molecule.

This assay set up has two main advantages that have not been exploited yet. First, the waveguide platform allows different capture molecules to be patterned so as to simultaneously detect different possible polymorphisms. Second, the SBEX assay makes wavelength multiplexing possible. Wavelength multiplexing uses different probes on the bases in order to simultaneously detect all base possibilities in a single channel. Combined, the exact identity of several polymorphisms can simultaneously be assayed with one channel of the waveguide.

### BRIEF DESCRIPTION OF THE DRAWINGS

FIG. 1. Single Base Extension on Planar Waveguides. Illustration of the planar waveguide biosensor employed in these studies showing evanescent field created by refracted light, extending ca. 110 nm from waveguide surface to excite bound fluorescent molecules.

FIG. 2. Cross-section of the flow cell used with the heat pump and heat sink.

FIG. 3. Reaction rate versus magnesium concentration. Solution: 10 mM Tris, pH 8.5, at 40° C.

FIG. 4. Reaction rates of each base and a water blank with probe DNA sequence exhibiting T at the point of polymerization. Solution: 10 mM Tris, pH 8.5, at 40° C.

FIG. 5. Reaction rate as a function of solution pH. Solution: 0.5 mM Tris, 0.5 mM MOPS, 0.5 mM HEPES, 0.5 mM MES, each with 2.5 mM MgCl2.

FIG. 6. Reaction rate versus DNA polymerase concentration at different Cy5-ddNTP monomer concentrations: top 1.3 nM, middle 0.65 nM, bottom 0.38 nM.

FIG. 7. Standard Curve of reaction rate versus concentration of DNA for determination of the sensitivity of the biosensor system. Conditions were 10 mM Tris, 10 mM MgCl₂, pH 8.5, and at 40° C. Reaction rates and oligonucleotide concentrations were plotted on logarithmic scales because of the large dynamic range of the data. The minimum detectable concentration was calculated at 30 pM.

FIG. 8. Temperature Dependency of Single Base Extension Reaction. Experiments were performed over a temperature range of 35-50° C. at a 5 U/mL L TPoly-I concentration. Three different dideoxynucleotide concentrations were examined: 0.4 nM (diamonds), 0.65 nM (circles), 1.3 nM (squares). The same analyte DNA concentration (100 pM) and buffer (10 mM Tris, pH 8.5, with 10 mM MgCl2) was used in all experiments. Data for each temperature was averaged with error bars showing standard deviation (n=3). A quadratic curve fit was used to determine the optimal reaction temperature for each dideoxynucleotide concentration: 0.4 nM (41.7° C.), 0.65 nM (41.1° C.), and 1.3 nM (41.3° C.).

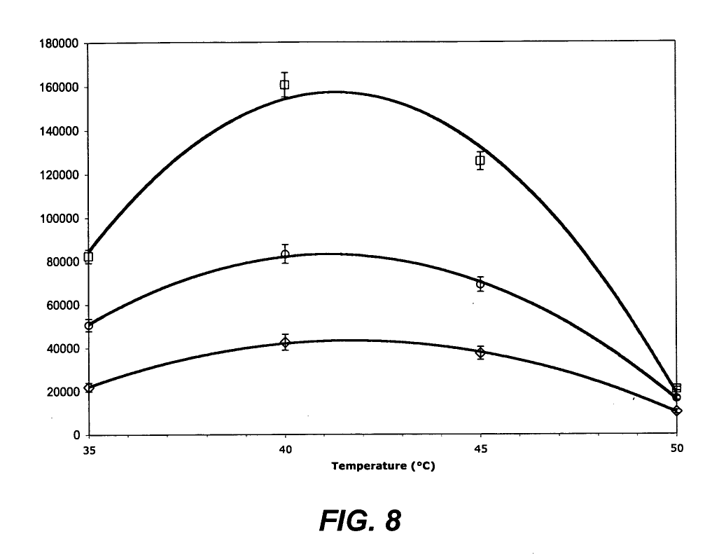

### DETAILED DESCRIPTION OF THE INVENTION

While this invention is described in certain embodiments and by way of certain examples, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

The assay system described herein combines single base extension (“SBEX”) with planar waveguide fluorescent biosensor technology to detect single nucleotide polymorphisms (SNP). Fifteen years ago, the Herron lab first investigated planar waveguides as fluorescent biosensors. See, e.g., Refs. 14-16. Since then, focus has been put on in vitro diagnostics (IVD) applications of this technology, particularly in critical care or point-of-care environments. In this context, immunoassays for analytes such as bovine serum albumin (BSA), human chorionic gonadotrophin (hCG), creatine phosphokinase isoform MB (CK-MB), and cardiac troponin I (cTnI) have been developed (4-10). More recently, planar waveguide fluorescence sensors have been used to monitor nucleic acid hybridization reactions (11, 12). Compared to conventional IVD assay technologies, planar waveguide fluorescence sensors offer the advantages of higher sensitivity, shorter assay times, multiple analyte determinations on a single specimen, internal calibration, and good performance with complex specimens such as serum, plasma, and whole blood (9).

SNPs are useful markers for gene diagnosis and mapping of genes on chromosomes. SNPs may be used to identify, inter alia, the presence of a disease, susceptibility to certain diseases and responsiveness to drug therapies. As will be recognized by a person of ordinary skill in the art, in light of the present disclosure, while SNPs are used to illustrate the invention, the invention may be applied to the analysis of any nucleotide change that results in a diagnostic or indicative change in at least one nucleotide.

Total internal reflectance fluorometry (TIRF) possesses two characteristics that are beneficial for fluorescent hybridization assays. The first is a selective excitation field (referred to as the “evanescent tail”) that, for practical purposes, only excites fluorescent molecules that hybridize to capture oligonucleotides immobilized within the evanescent tail. The amplitude of the wave in the evanescent tail decreases exponentially with increasing distance from the interface surface, decaying over a distance of about one light wavelength. The second is the ability to take kinetic measurements, which allow greater precision, provide information about the shape of the hybridization curve, and are insensitive to change in fluorescence between assay runs. Real time detection provides greater sensitivity than end point assays, which allows for fewer cycles of amplification.

In traditional DNA hybridization assays using TIRF assays detection, an optical substrate (such as a planar waveguide) immobilizes oligonucleotides that bind to their complementary strands of DNA. The evanescent wave generated in the waveguide substrate will only excite fluorescently labeled analyte DNA molecules that have bound to the stationary capture oligonucleotides. This evanescent excitation increases the assay speed by eliminating wash and reagent addition steps, which increase the speed and throughput of the assays.

The dept of the evanescent wave which is useful for measurements is within about 300 nm of the sensor surface.

One fluorescent affinity type assay is single base extension (SBEX). SBEX uses a DNA polymerase to incorporate Cy5 labeled dideoxynucleotriphosphates (ddNTPs). The label, Cy5, is an example of a fluorescent label used for detection. Additional labels include, but are not limited to, Cy3, Cy3.5, Cy5.5, Cascade Blue-7, BODIPY, Alexa Fluor, Oregon Green, Fluorescein, Rhodamine Green, Tetramethylrhodamine, and Texas Red (see, for example, Molecular Probes, handbook, available on line at probes.com). The ddNTP monomer lacks the 3′ OH, which is beneficial in terminating the polymerase reaction so that only one base is added. Each base added by the polymerase is complementary to the corresponding position on the stationary capture molecule. SBEX, which compares affinity one base at a time, has better selectivity than a traditional affinity assay, which compares the affinity of a group of bases (17-21 bases) at the same time.

Identification (“calling”) of the single base added to the 3′ end of the probe molecule can be done in one of three ways: parallel channels for each of the four bases using a different labeled ddNTP in each channel; sequential SBEX reactions using a different labeled ddNTP in each reaction; or wavelength discrimination of the four possibilities using a different fluorescent label for each ddNTP. The first of these methods may be preferred. SBEX may be used in oligonucleotide genotyping and SNP detection systems, and is advantageous over traditional hybridization assays, for example, due to greater base specificity, production of a covalent bond between the labeled ddNTP and the probe, and simultaneous detection of multiple bases.

By using SBEX on waveguides, simultaneous detection of several different polymorphisms can be done with ease. By patterning the waveguide with different capture sequences, different points in a sequence, for example, a genome, a chromosome and/or a gene, may be assayed. As SBEX only requires a fluorescent label on the ddNTP monomers used, all instances of a particular base will be detected. In order to do the same thing with a traditional DNA hybridization assay, each probe DNA for each capture sequence would have to be fluorescently labeled.

The enzyme-catalyzed reaction has two distinct advantages. First, a stable covalent bond forms between the stationary phase and a labeled monomer, e.g., a Cy5-labeled monomer. This increases the assay sensitivity versus traditional hybridization assays where the fluorescent label is captured by the stationary phase via non-covalent interactions (duplex formation). Although an embodiment, used herein to illustrate the invention, does not require a washing step, other embodiments further exploit the covalent bond stability to enable a stringent washing step. Second, the polymerase enzyme incorporates the dideoxynucleotide with high fidelity—due to the replication accuracy of a polymerase, in general only the base that is complementary to the target base will react. SBEX is particularly well suited for planar waveguide technology, benefiting from the increased speed of a washless assay and increased sensitivity provided by kinetic data.

Using SBEX on the waveguide platform, enables a rapid assays (<5 min) results to be performed that is are able to differentiate between single nucleotide polymorphic and wild type sequences at temperatures less than 50° C.

Fluorescence imaging is sensitive to speed, sensitivity, noise and resolution, and each may be optimized for use in the invention, for example, speed may be increased to increase assay times. As will be recognized by a person of ordinary skill in the art each factor may influence another factor, for example, speed typically affects sensitivity, since the signal is affected by the exposure duration. In addition, sensitivity is the ability to detect an event and record a detectable signal.

Base extension may be detected using a CCD camera, a streak camera, spectrofluorometers, fluorescence scanners, or other known fluorescence detection devices, which generally comprise four elements, an excitation source, a fluorophore, a filter to separate emission and excitation photons, and a detector to register emission photons and produce a recordable output, typically an electrical or photographic output.

Polymerase enzymes useful in the invention are known in the art and include, but are not limited to, thermostable polymerases, such as pfu, Taq, Bst, Tfl, Tgo and Tth polymerase, DNA Polymerase I (E.C. 2.7.7.7), Klenow fragment, and/or T4 DNA Polymerase. The polymerase may be a DNA-dependent DNA polymerase, a DNA-dependent RNA polymerase, a RNA-dependent RNA polymerase, a RNA-dependent DNA polymerase or a mixture thereof, depending on the template, primer and NTP used. The polymerase may or may not have proofreading activity (3′ exonuclease activity) and/or 5′ exonuclease activity.

The capture molecule and/or the analyte molecule of the invention may be any nucleic acid, including, but not limited to, DNA and/or RNA and modifications thereto known in the art, and may incorporate 5′-O-(1-thio)nucleoside analog triphosphates, α-thiotriphosphate, 7-Deaza-α-thiotriphosphate, N6-Me-α-thiotriphosphate, 2′-O-Methyl-triphosphates, morpholino, PNA, aminoalkyl analogs, and/or phosphorotioate.

The present invention allows for the diagnosis of any disease having a polymorphism that distinguishes the disease state from the healthy state, including, but not limited to, the following diseases (genes associated with the disease state or increased probability of acquiring the disease state): congenital adrenal hyperplasia (steroid 21-hydroxylase, CYP21), breast cancer (BRAC1, BRAC2), Chinese Spring wheat, Alzheimer's disease (CYP46, SOAT1), inherited thrombophilia (FV R506Q), Hereditary Hemochromatosis (HFE G845A), increased risk factor for venous thrombosis (prothrombin), Hereditary Non-Polyposis Colorectal Cancer (hMSH2, hMLH1, APC), acute promyelocytic leukemia (t(15;17)(q22;q21)), Graves' disease (TSHR), beta-thalassemia, abnormal hemoglobins, type 2 diabetes (PPARγ), and other diseases or predispositions to disease. The present invention may also be used for applications such as paternity testing, (see, M. P. Weiner and T. J. Hudson, (2002) Introduction to SNPs: Discovery of Markers for Disease, BioTechniques 32: S4-S13).

The invention is further explained with the aid of the following illustrative examples.

### Experimental

DNA Synthesis and Purity

The LQTS SNP of interest in the present Example was the G760A polymorphism. A 5′-biotinylated probe oligonucleotide was synthesized for detection. It was 21 nucleotides in length with the following sequence:

| | |
|---|---|
| Probe Oligonucleotide: | (SEQ ID NO:1) |
| 5′-biotin-CCTGGCGGAGGATGAAGACCA-3′ | |

Synthetically made oligonucleotides (29-mers) were used to demonstrate the application of the technology to real PCR analyte DNA derived from a human patient. Synthetic oligonucleotides were designed to detect and differentiate between the wild type sequence and the G760A polymorphism. Model analyte oligonucleotides were 29 nucleotides in length, with the middle 21 bases complementary to the probe sequence. Four bases extended on both sides beyond the probe DNA, with the fourth base (first to be polymerized) varying in each analyte sequence:

| | |
|---|---|
| Analyte G (Wild Type): | |
| 5′-TCCGTGGTCTTCATCCACCGCCAGGAGCT-3′ | (SEQ ID NO:2) |
| Analyte A (G760A): | |
| 5′-TCCATGGTCTTCATCCACCGCCAGGAGCT-3′ | (SEQ ID NO:3) |
| Analyte C: | |
| 5′-TCCCTGGTCTTCATCCACCGCCAGGAGCT-3′ | (SEQ ID NO:4) |
| Analyte T: | |
| 5′-TCCTTGGTCTTCATCCACCGCCAGGAGCT-3′ | (SEQ ID NO:5) |

Oligonucleotides were synthesized by a peptide and nucleic acid synthesis facility at the University of Utah. All products were then purified using HPLC to remove the excess salts and the “n-1” oligonucleotides that had a base deletion.

Planar Waveguide Fluorescent Biosensor

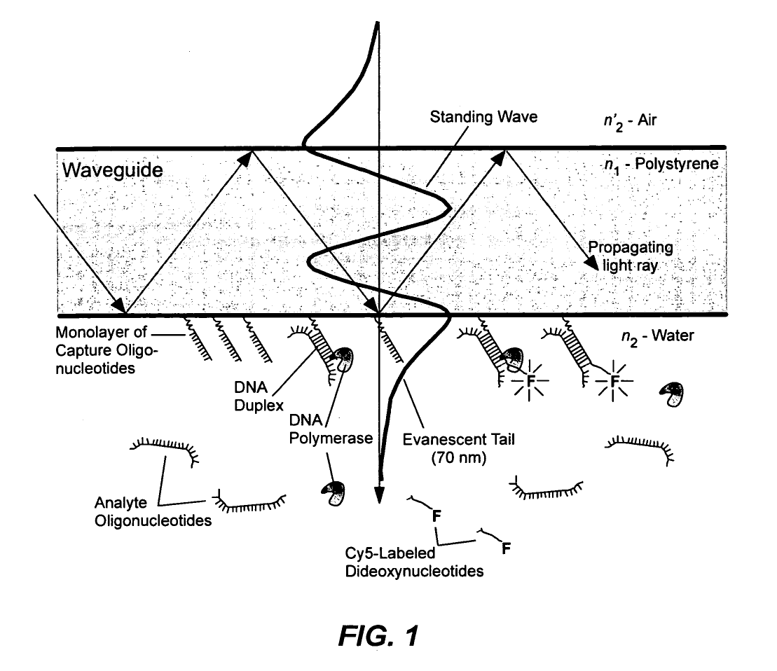

The planar waveguide fluorescent biosensor system used in these studies is that described in U.S. Pat. Nos. 5,512,492 and 5,516,703 (14-16). Injection-molded planar waveguide sensors were fabricated from polystyrene by Opkor, Inc. (Rochester, N.Y.). The sensors consisted of a 25×25×0.5 mm planar waveguide and a light coupling lens (inclined at ca. 20° to the plane of the waveguide) both molded into a single piece. The light source was a 15 mW semiconductor laser that emitted at 638 nm or 635 nm. The laser light was formed into a sheet beam (20mm×1mm) and coupled into the waveguide via the integrated coupling lens. Once coupled, the light propagated the length of the waveguide exhibiting total internal reflection between the upper and lower faces of the waveguide. Constructive interference at each reflection produces a transverse standing wave within the waveguide that exponentially decays into the surrounding medium producing an evanescent tail (see, FIG. 1). This standing wave did not have a sharp boundary at the waveguide surface, but instead decayed exponentially as it penetrated into the surrounding medium. The rate of decay into the medium depended on the indexes of refraction of the waveguide and the medium, the angle of incidence and the wavelength of the light. It is calculated that with the current setup the evanescent field retained enough intensity to excite a fluorescent molecule 110 nanometers from the surface.

The format of the present assay consisted of a single-stranded “capture” oligonucleotide immobilized to the planar waveguide, soluble single-stranded “analyte” oligonucleotide, a Cy-5 labeled ddNTP monomer, and a polymerase enzyme (see, FIG. 1). Analyte DNA diffused through the bulk solution and hybridized with immobilized capture oligonucleotides. Once hybridized, the DNA polymerase enzyme can bind to the double helix. The polymerase enzyme polymerizes the capture oligonucleotide with the base complementary to the point of possible polymorphism. If the polymerized base was labeled with Cy-5, it will be excited by the evanescent field and fluoresce to give a signal. (Although the size of the duplex DNA varied with analyte size, it was generally smaller than the penetration depth (ca. 110 nm) of the evanescent field of the sensor system.)

In one embodiment, a flowcell was used to partition the waveguide into three independent detection zones (channels).

The fluorescent signal is collected by a CCD camera (Model ST-6, Santa Barbara Instruments Group) through an interference filter (670 nm center wavelength, 40 nm bandpass, Omega). Data from each pixel in the CCD camera are collected at 10-second intervals over a 5-minute period using LabView® to produce an independent binding kinetics plot for each channel. The signal was collected through an interference filter (Center wavelength 670 nm, pass band 40 nm, Omega) and was detected by a CCD camera (Model ST-6, Santa Barbara Instruments Group) oriented such that its collection axis was normal to the plane of the waveguide.

Immobilization of Capture Oligonucleotides

Capture oligonucleotides were immobilized as described by Herron et al (11). Clean, dry waveguides were first coated for 1 hour with a 150 nM solution of neutravidin in phosphate buffered saline (50 mM PBS, pH 7.5, 100 mM NaCl, with 0.02% sodium azide as a preservative). Unadsorbed neutravidin was removed by twice washing each waveguide in TE buffer (10 mM Tris, 1 mM EDTA, pH 7.4). Then, a 50 nM solution of 5′-biotinylated oligonucleotide in TE buffer was allowed to react with the immobilized neutravidin for 1 hour, followed by washing twice with TE buffer. Finally, waveguides were coated for 30 minutes with a 0.1% (w/v) solution of trehalose in TE buffer. Excess solution was poured off, and the remaining trehalose was allowed to dry for several hours at 4° C. This final post-coating step protected the immobilized oligonucleotides and allowed coated waveguides to be stored at room temperature for more than one month.

Cleaning and Recycling of Waveguides

In order to decrease cost of each assay, the waveguides were recycled after use. A 5% Clorox® bleach solution was chosen (12), as this cleaning solution exhibited the best tradeoff between damaging and cleaning the surface of the waveguide. The waveguides were washed with water three times then soaked in 5% Clorox® overnight. Waveguides were again washed with water several (approximately 5) times, and allowed to dry before re-coating. Approximately 5-10% of the waveguides were scratched or cracked by the biosensor or washing and were discarded after examination.

Flowcell and Reaction Chamber

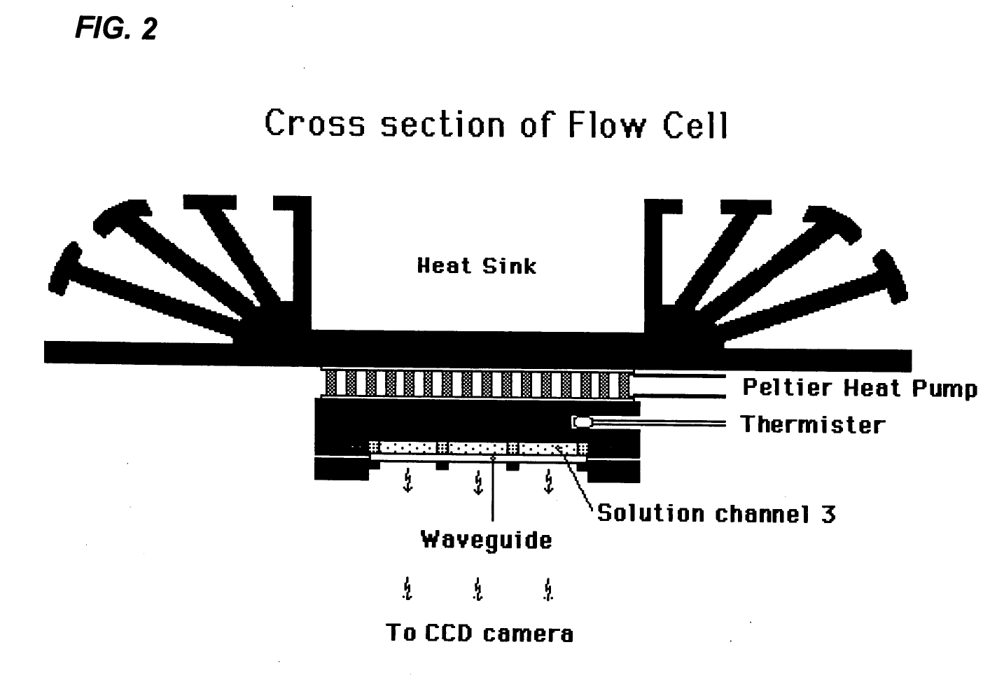

The flow cell held the waveguide in place and provided three chambers for the injected solutions, each of which was in contact with approximately one-third of the coated waveguide. These chambers were treated independently in data collection and solution choice, but all had the same capture oligonucleotide coating. A different solution of analyte DNA and ddNTP may be injected into each channel and each channel may have one or more capture molecules. FIG. 2 shows the cross-section of the flow cell illustrating the three channels.

The temperature of the reaction chamber was controlled by a Peltier heat pump connected to a heat sink, as shown in FIG. 2. The temperature was monitored by a thermister imbedded in the flow cell body.

Experimental Analysis

The effects of reaction temperature were examined in a series of experiments in which temperature was varied over a range of 25-60° C. at varied reactant conditions (TPoly-I 10-50 units/mL, Cy5-ddNTP 1.3-13 nM).

Initial experiments were performed using reagent concentrations (1.3 nM of ddNTP, and 10 units/mL of TPoly-I) recommended by the commercial sequencing kit (Amersham Biosciences) over a temperature range of 25-60° C. Subsequent experiments were performed at reduced polymerase, TPoly-I, concentrations (1.25 units/mL, 2.5 units/mL, 5 units/mL) coupled with reduced Cy5-ddNTP concentrations (0.33 nM, 0.65 nM at temperatures of 30-50° C. The same analyte DNA concentration (100 pM) and buffer (10 mM Tris, pH 8.5, with 10 mM MgCl₂) was used in all experiments. Factors such as initial reaction rate, reaction rate after 5 minutes, reaction time (5 vs. 16 minutes), and reproducibility were examined.

The effects of pH were examined in a series of experiments monitoring the 5-minute reaction rate in which pH was varied over a range of 6.5 to 10.5. A mixture of Good buffers (Tris, MOPS, HEPES, and MES: 0.5 mM of each) was used to ensure adequate buffer capacity. The concentration of magnesium ions was maintained at 2.5 mM to prevent the precipitation of magnesium hydroxide. Solutions were prepared from a pH 10.5 stock solution by adding 0.1 M HCl to lower the pH to the desired point.

The effect of magnesium concentration on reaction rate was explored by varying MgCl₂ concentration between 0.67 mM and 10 mM and monitoring reaction rate. Assays were run in 10 mM Tris pH 8.5 buffer, at 40° C.

Minimum detection limit (MDL) was determined from a standard curve of SBEX reaction rate versus bulk analyte DNA concentration. A 95% upper confidence limit for MDL was calculated by dividing twice the standard deviation of the blank by the slope of the standard curve at low DNA concentrations. MDL was determined for two different solutions—the standard buffer solution used in most experiments (10 mM MgCl₂, 10 mM Tris, pH 8.5) and the solution used for the pH analysis (2.5 mM MgCl₂, 0.5 mM Tris, 0.5 mM HEPES, 0.5 mM MOPS, 0.5 mM MES, pH 8.5), which contained several buffers and reduced MgCl₂ to prevent precipitation of magnesium hydroxide [20, 21, 26-28].

As will be understood by a person of ordinary skill in the art, the required magnesium (a polymerase cofactor) in the solution may activate DNA digesting pyrogens that may be in the solution. Hence, in one embodiment, the sample may be treated to remove pyrogens or to inhibit such pyrogens. For example, DNase and/or RNase inhibitors, e.g., Aurintricarboxylic acid, sodium citrate, angiogenin-binding protein, may be added to the sample, optionally the sample may be treated with proteases to degrade pyrogens and treated to remove and/or inhibit proteinase activity, e.g., by heat treatment and/or pepstatin A, leupeptin, phenylmethyl sulfonyl fluoride (PMSF), and/or aprotinin.

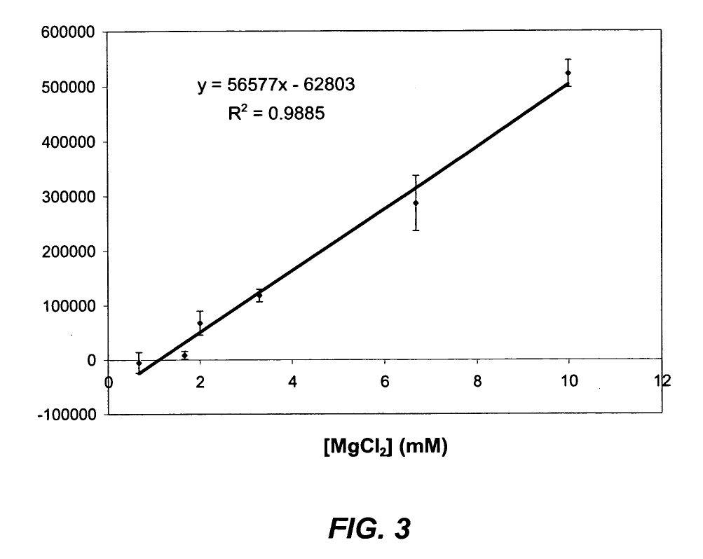

Like many other polymerases, TPoly-I requires magnesium for activity. FIG. 3 shows the reaction rate dependence of TPoly-I polymerase on magnesium concentration. Experiments were performed over magnesium concentrations of 0.67 mM to 10 mM. The reaction rate increased linearly over a concentration range of 2-10 mM. Below 2 mM the signal was not statistically significant. Both TPoly-I activity and DNA hybridization are facilitated by magnesium ions. The linear dependence of the reaction rate indicates that TPoly-I activity, is rate limiting.

Results and Discussion:

Advantages of Waveguide

The planar waveguide technology has two main advantages: it provides an evanescent field and a translucent stationary phase for immobilization of nucleic acid probes. The evanescent wave has the ability to selectively excite fluorescent molecules that are in very close proximity to the surface. This effect will excite only molecules that are bound to the surface, eliminating the washing step in hybridization like assays (see, FIG. 1). This saved significant time and minimized false positive signals. In combination with a translucent the stationary phase, it is also possible to detect hybridization events with fluorescent molecules in real time by detecting the emitted light from across the waveguide (see, FIG. 2). The second advantage, shared by all arrayed planar sensors, is a stationary phase that can be patterned for better detection limits, or arrayed for simultaneous detection of multiple possible polymorphisms.

A third advantage of using SBEX is the ability to detect several base identities simultaneously. By labeling the different bases with different fluorescent labels, each base can be assayed independently in the same reaction chamber.

Advantages of Kinetic Measurements Over End-Point Collection

Kinetic measurements allowed a greater degree of precision than a single end point measurement. Kinetic measurements provide information about the shape of the hybridization curve and are insensitive to the native fluorescence change between waveguides. The data collection procedure (described herein) had a sampling period of 10 seconds, which allowed 6 data points to be collected per minute. Although not continuous, this was adequate for monitoring hybridization kinetics in the subnanomolar concentration range that was used in these studies. Kinetic measurements also provided information about the shape (i.e., kinetics profile) of the hybridization curve, which could be exploited to detect mismatched bases in duplex DNA. For instance the shape of the curve can indicate the degree of completeness of the reaction. Finally, kinetic measurements were inherently insensitive to the change in native fluorescence generated by the polystyrene of the waveguide, thus decreasing extra-assay variability.

Minimum Assay Time

Intuitively, the reaction rate should be proportional to reactant concentration with the highest rate occurring at the beginning of the assay (because of depletion of reactants at longer times). However, the maximum SBEX reaction rate was delayed 30-60 seconds from the start of the reaction. This delay suggested the presence of a rate-limiting step, a premise that was examined in a series of pre-wetting experiments. In the first experiment the waveguide was pre-wet with the analyte DNA, allowing double helices to form before the TPoly-I and Cy5-ddNTPs were introduced. The second experiment pre-wet the waveguide with analyte DNA and TPoly-I allowing double helices to form and the polymerase enzyme to bind before the Cy5-ddNTPs were introduced. Only after pre-wetting with DNA and TPoly-I did the lag disappear, indicating that either the diffusion or binding of the polymerase was the rate-limiting step.

The lag time observed in the standard protocol (simultaneous addition of analyte DNA, TPoly-I and Cy5-ddNTP) may place a lower boundary of about 5 minutes on assay time. Pre-wetting with DNA and TPoly-I does eliminate lag time, although, the extra reagent addition step also adds to assay time and may also increases assay complexity. Therefore, in a point-of-care setting, depending on the sophistication of the particular point-of-care facility, it may be desirable to increase the assay time. However, the invention allows the facility to adjust the assay so as to achieve the appropriate assay time and complexity.

Specificity

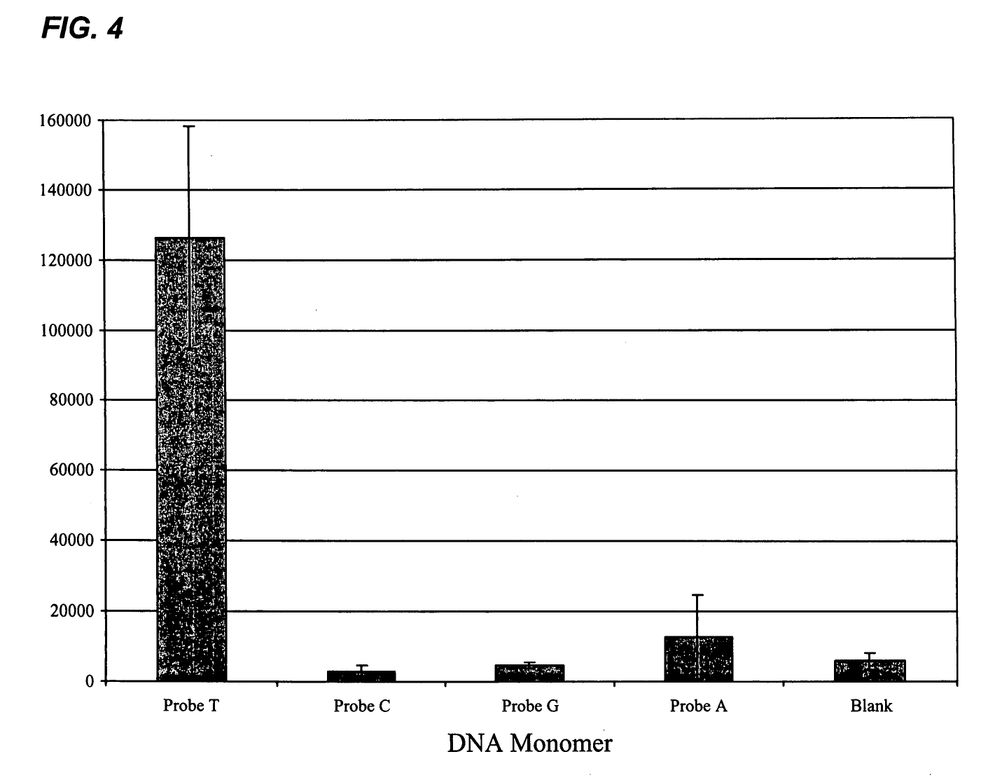

To accurately detect a SNP, only the complementary base should be incorporated to the 3′ end of the capture sequence by the polymerase enzyme. All the other bases should not be incorporated. Several experiments were done with the probe DNA that corresponded to the G760A polymorphism, which had a C at the point of possible polymorphism. All ddNTPs were tried against this sequence, of which only ddGTP should be incorporated. The non-complementary bases resulted in a signal that was no greater than the water blank while the complementary base gave a good signal (see, FIG. 4). Hence, rejection of non-complimentary ddNTPs was very good.

The SBEX assay format used to illustrate the invention consists of a single-stranded “capture” oligonucleotide probe immobilized to the planar waveguide and a sample containing the single-stranded analyte oligonucleotide, a Cy-5 labeled ddNTP monomer, and TPoly-I (see FIG. 1). Injecting the sample into the flowcell initiates the reaction. Analyte DNA diffuses through the bulk solution and hybridizes with the immobilized capture oligonucleotide, followed by binding of TPoly-I to the double helix [29]. TPoly-I incorporates the complementary Cy5-labeled ddNTP at the 3′ end of the capture oligonucleotide at the position of the suspected polymorphism. Upon incorporation, the dye will be excited by the evanescent field and emit a signal; otherwise, only background signal is generated. TPoly-I was chosen for these studies because it exhibits a high incorporation rate for Cy5-labeled ddNTPs. Other polymerases incorporate Cy5 labeled ddNTPs several thousand times slower [26], but, as will be understood by a person of ordinary skill in the art in light of the present invention, may be used for non-labeled deoxynucleotriphosphates (dNTPs), non-labeled ddNTPs, dNTPs or ddNTPs labeled with alternative fluorescent molecules and/or wherein the decreased incorporation rate is acceptable.

To assay for the specific G760A single nucleotide polymorphism, a solution containing the analyte DNA, the polymerase enzyme (TPoly-I), and at least one labeled nucleotide (e.g., Cy5-ddNTP) was injected into each channel of the 3-channel flowcell. A signal was produced if the suspected polymorphism was complementary to the particular Cy5-ddNTP added to a given channel. Channel one contained Cy5-ddCTP, to detect G at position 760, the wild type sequence. Channel two contained Cy5-ddTTP, to detect A, the SNP sequence. Channel three contained Cy5-ddGTP and Cy5-ddATP, to detect both C and T. In the case of wild type DNA, only channel one will produce a signal. In the case of heterozygous SNP DNA, both channel one and two will produce a signal. Channel three acts as an error signal, only producing a signal if there is either a systemic error, or the DNA sequence contained an undocumented polymorphism at position 760: C or T.

The fidelity of the reaction was examined in several experiments. Each analyte sequence (G, A, C, and T) was tested against all possible Cy5-ddNTPs monomers, and a water blank as background. Table 3 shows the relative reaction rate of each Cy5-ddNTP to four different analyte DNA sequences, each containing a different base at position 760. A one-tailed Student's T-test (P=0.05) was employed to determine if any of the reactions were significantly greater than background. The P-values from this T-test are shown in Table 3. Only the complementary ddNTP to each analyte DNA was significantly greater than the background.

TABLE 3
SBEX Rate for each analyte base sequence with each ddNTP base
possibility.

| | Base in Analyte DNA | | | | | | | | | | | |
|---|---|---|---|---|---|---|---|---|---|---|---|---|
| | Analyte G (WT) | | | Analyte A (G760A) | | | Analyte C | | | Analyte T | | |
| ddNTP | Rate¹ | Std² | Prb³ | Rate¹ | Std² | Prb³ | Rate¹ | Std² | Prb³ | Rate¹ | Std² | Prb³ |

| | | | | | | | | | | | | |
|---|---|---|---|---|---|---|---|---|---|---|---|---|
| C | 1.000* | 0.302 | 0.02 | 0.036 | 0.007 | 0.21 | 0.024 | 0.026 | 0.96 | 0.010 | 0.002 | 0.79 |
| T | 0.014 | 0.025 | 0.79 | 1.000* | 0.251 | 0.01 | 0.028 | 0.009 | 0.96 | 0.016 | 0.007 | 0.93 |
| G | 0.049 | 0.003 | 0.07 | 0.067 | 0.070 | 0.34 | 1.000* | 0.258 | 0.01 | 0.018 | 0.032 | 0.70 |
| A | 0.008 | 0.022 | 0.87 | 0.024 | 0.011 | 0.90 | 0.116 | 0.023 | 0.77 | 1.000* | 0.205 | 0.01 |
| Background | 0.030 | 0.012 | | 0.047 | 0.018 | | 0.149 | 0.059 | | 0.038 | 0.015 | |

¹SBEX rates were normalized to complementary base pair. Reaction rates that are significantly greater than the background (probability value below 0.05) are noted with (*).
²Std: standard deviation.
³Prb: probability value, generated by a 1-tailed T test compared to the background rate.

Optimization Curves:

Reaction temperature is of primary consideration because high temperatures can increase reaction kinetics resulting in faster assays. However, such gains can be undone by the longer solution incubation times required at elevated temperature. Also, helix formation will be destabilized at temperatures in excess of Tₘ, totally abrogating the reaction. Experiments were performed over a temperature range of 25-60° C. at different reactant concentrations (TPoly-I 10-50 units/mL, Cy5-ddNTP 1.3-13 nM). Since the reaction rate varies with both solution conditions and temperature, it was normalized for each set of solution conditions for comparison of temperature effects independent of solution effects. Maximum reaction rate was used as a normalization parameter within each solution. FIG. 2 shows the average normalized reaction rate over the temperature range. The maximum reaction rate occurred at 41.2° C. as determined by the second-order polynomial curve fit shown in the FIG. 2. It was found that the optimum temperature was between 40 and 45° C. All subsequent assays were done at 40° C. All data presented in FIG. 3 are for 5-minute assays, longer assay times or endpoint assays may exhibit a different temperature maximum.

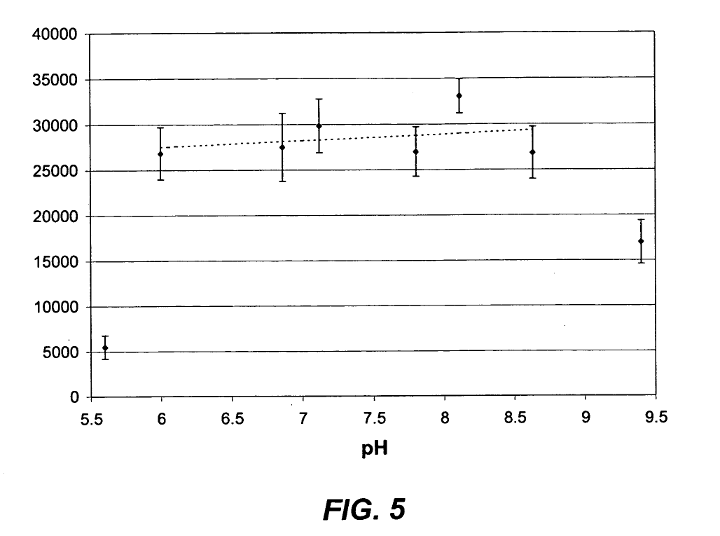

Solution pH can affect TPoly-I activity, as well as the enzyme's affinity for duplex DNA. Although the pH optimum for TPoly-I has not been determined, most experimental procedures using this enzyme specify a pH of around 8.5 [20, 21, 26-28]. The experimental results shown in FIG. 5 show the reaction rate of as a function of solution pH. In order to change the pH over the desired range, a mixture of Good buffers was used: Tris, MOPS, HEPES, and MES: 0.5 mM of each. In order to prevent the precipitation of magnesium hydroxide, the concentration of magnesium ions was kept at a lower concentration for the pH study than for the other experiments: 2.5 mM. The reaction rate is fairly uniform over a large pH range. The reaction rate does show a decrease at either end of the pH range (above 10.5, below 5.5). The pH chosen for other experiments was 8.5. This was because of the simplicity of buffer solution preparation (only tris is needed) and solubility of magnesium ions.

Like other polymerases, Thermo Sequenase Polymerase needs magnesium ions to be active. FIG. 3 shows that the reaction rate was dependent upon the concentration of magnesium ions. Experiments were done at magnesium concentrations from 0.67 mM to 10 mM. The reaction rate increased linearly with magnesium concentration in the range of 2 mM and 6.67 mM. Below 2 mM there was no significant reaction signal. Above 6.67 mM there was slight increase in reaction rate, but it seems to be nearing a plateau.

Assay optimization required consideration of two competing factors—assay speed and sensitivity versus cost. In particular, high concentrations of Cy5-ddNTP and/or TPoly-I increase speed and/or sensitivity, but typically also increase assay cost. The effects of ddNTP and TPoly-I concentrations on 5- and 16-minute reaction rates are shown in Tables 1 & 2, respectively. For the 5-minute reaction (Table 1), the 2.5, 5 & 10 U/mL TPoly-I data sets exhibited pre-saturation Michaelis-Menton behavior with the SBEX reaction rate increasing linearly with ddNTP concentration. For the 16-minute reaction (Table 2), the 2.5, 5, 100 U/mL TPoly-I data sets all appeared to saturate at the highest ddNTP concentration (1.3 nM). Such non-linearities are usually due to depletion of either substrate or enzyme, or to the accumulation of product, all three of which are more pronounced at longer reaction times. The data suggest that enzyme depletion may be responsible for the observed saturation.

The 5-minute reaction rate for the 1.25 U/mL TPoly-I concentration was indistinguishable from background at the two higher ddNTP concentrations, this result is believed to be due to pipetting errors that can occur when adding small amounts of enzyme to the reaction mixture. The 1.25 U/mL TPoly-I data set for the 16-minute reaction (Table 2) is also believed to be due to the same reason.

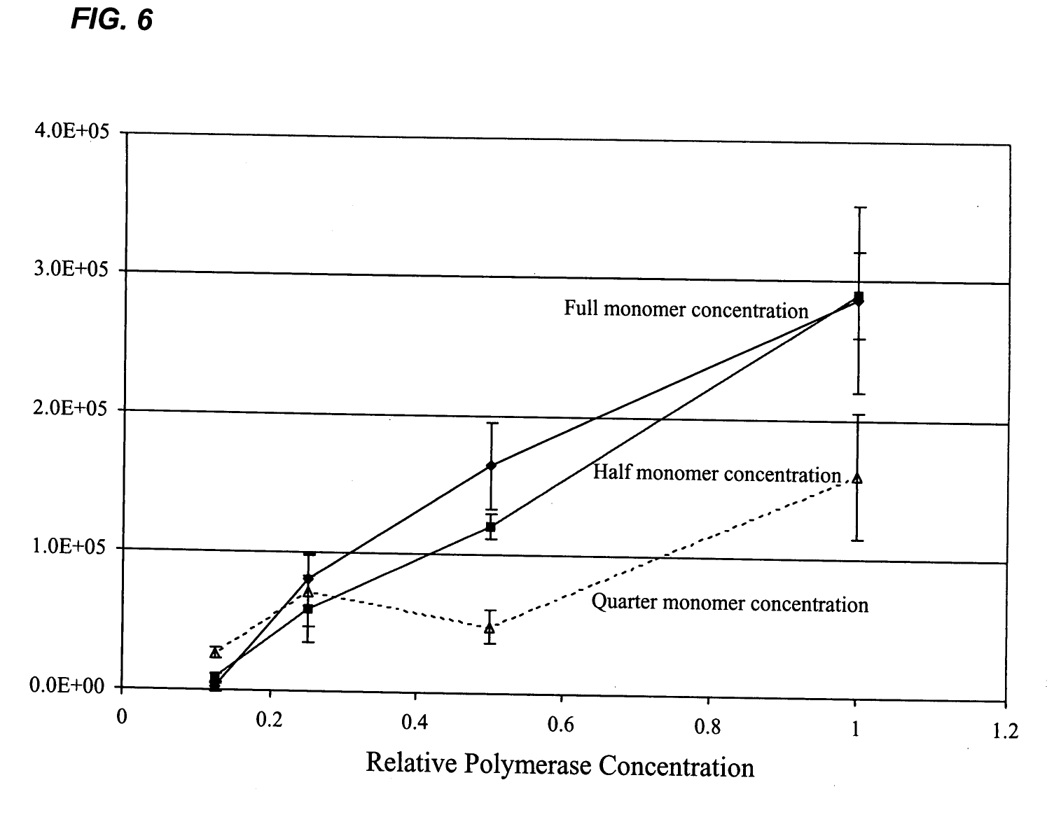

Based on these results, the highest TPoly-I concentration (10 U/mL) was selected for subsequent experiments because of its good linearity in both the 5- and 16-minute reactions. The highest (1.3 nM) ddNTP concentration was also chosen in order to maximum reaction rate, thereby improving signal-to-noise ratio. The reaction rate as a function of ddNTP and polymerase concentration is shown in FIG. 6. The optimum concentrations were between 0.65 nm and 1.3 nM of ddNTP, and 10 units/ml of the polymerase.

TABLE 1
Effects of ddNTP monomer and TPoly-I polymerase concentrations on
single base extension (SBEX) rate over a 5-minute reaction period.¹

| | SBEX Reaction Rate × 10⁻³ | | | |
|---|---|---|---|---|
| | (AU/min) | | | |
| TPoly-I Concentration | ddNTP Concentration | | | Linearity² |
| (U/mL) | 0.33 nM | 0.65 nM | 1.3 nM | (R²) |
| 1.25 | 26.5 | Ind² | Ind³ | ND⁴ |
| 2.5 | 44.3 | 58.9 | 80.3 | 0.993 |
| 5 | 82.2 | 120 | 164 | 0.978 |
| 10 | 160 | 217 | 287 | 0.982 |

¹Reaction conditions: 10 mM MgCl₂, 10 mM Tris, pH 8.5, 40° C.
²Linearity was estimated by computing the correlation coefficient (R²) between SBEX reaction rate and ddNTP concentration at each TPoly-I concentration.
³Indinstinquishable from background.
⁴Not Determined.

TABLE 2
Effects of ddNTP monomer and TPoly-I polymerase concentrations on
single base extension (SBEX) rate over a 16-minute reaction period.¹

| | SBEX Reaction Rate × 10⁻³ | | | |
|---|---|---|---|---|
| | (AU/min) | | | |
| TPoly-I Concentration | ddNTP Concentration | | | Linearity² |
| (U/mL) | 0.33 nM | 0.65 nM | 1.3 nM | (R²) |
| 1.25 | 73 | 22 | 165 | 0.596 |
| 2.5 | 128 | 197 | 223 | 0.811 |
| 5 | 227 | 332 | 333 | 0.576 |
| 10 | 360 | 561 | 659 | 0.855 |

¹Reaction conditions: 10 mM MgCl₂, 10 mM Tris, pH 8.5, 40° C.
²Linearity was estimated by computing the correlation coefficient (R²) between SBEX reaction rate and ddNTP concentration at each TPoly-I concentration.

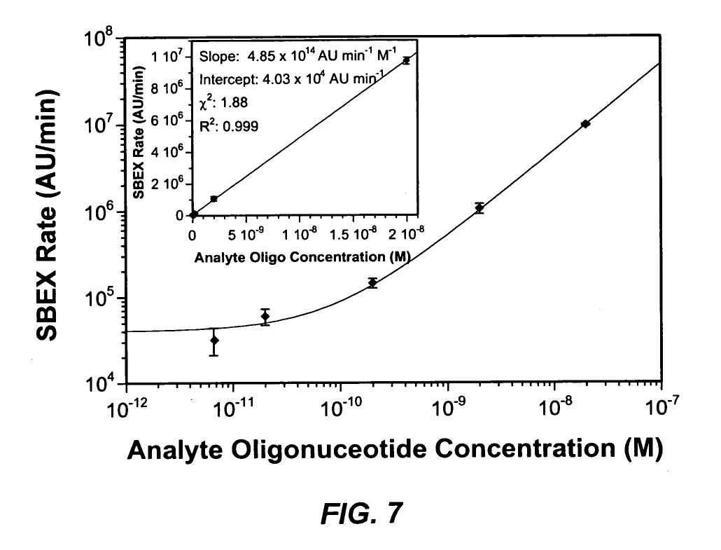

The standard curve for calculation of the detection limit is shown in FIG. 7. The curve resembles a classic Mechelis-Menton binding curve. The detection limit was determined by calculating the concentration that would give a reaction rate equal to the blank signal plus twice its standard deviation. Using this procedure, the detection limit was calculated to be 30 pM for a 5 minute assay, and 12 pM for a 16 minute assay (data not shown), both at 10 mM MgCl2. The detection limit was also calculated using the 2.5 mM MgCl2 solution, used for the pH study, was found to be around 100 pM. This diminished detection limit may be a result of the lower magnesium concentration, activating less of the DNA polymerase.

All references, including publications, patents, and patent applications, cited herein are hereby incorporated by reference to the same extent as if each reference were individually and specifically indicated to be incorporated by reference and were set forth in its entirety herein.

### REFERENCES

The following references are incorporated by this reference in their entirety.
1. Splawski, I., Tristani-Firouzi, M., Lehmann, M. H., Sanguinetti, M. C., and Keating, M. T. (1997) Nature Genetics 17, 338-340.
2. Wang, Q., Shen, J., Splawski, I., Atkinson, D., Li, Z., Robinson, J. L., Moss, A. J., Towbin, J. A., and Keating, M. T. (1995) Cell 80, 805-811.
3. Selvakumar, N., Ding, B.-C. D., and Wilson, S. M. (1997) BioTechniques 22, 604-606.
4. Ives, J. T., Reichert, W. M., Lin, J.-N., Hlady, V., Reinecke, D., Suci, P. A., Van Wagenen, R. A., Newby, K., Herron, J., Dryden, P., and Andrade, J. D. (1987) in Optical Fiber Sensors (Chester, A. N., Ed.), pp. 391-397, M. Nijhoff, Dordrecht.
5. Christensen, D., Dyer, S., Fowers, D., and Herron, J. (1993) in Fiber Optic Sensors in Medical Diagnostics, Vol. 1886, pp. 2-8, SPIE, Los Angles, Calif.
6. Herron, J. N., Caldwell, K. D., Christensen, D. A., Dyer, S., Hlady, V., Huang, P., Janatova, V., Wang, H.-K., and Wei, A.-P. (1993) in Advances in Fluorescence Sensing Technology, Vol. 1885, pp. 28-39, SPIE, Los Angles, Calif.
7. Christensen, D. A., and Herron, J. N. (1996) in Ultrasensitive Biochemical Diagnostics, Vol. 2680, pp. 58-67, SPIE, San Jose, Calif.
8. Plowman, T. E., Reichert, W. M., Peters, C. R., Wang, H. K., Christensen, D. A., and Herron, J. N. (1996) Biosens Bioelectron 11, 149-60.
9. Herron, J. N., Wang, H.-K., Terry, A. H., Durtschi, J. D., Tan, L., Astill, M. E., Smith, R. S., and Christensen, D. A. (1998) in Systems and Technologies for Clinical Diagnostics and Drug Discovery, Vol. 3259, pp. 54-64, The International Society for Optical Engineering, San Jose, Calif.
10. Plowman, T. E., Durstchi, J. D., Wang, H. K., Christensen, D. A., Herron, J. N., and Reichert, W. M. (1999) Anal Chem 71, 4344-52.
11. Herron, J. N., S. zumBrunnen, J.-X. Wang, X-L. Gao, H.-K. Wang, A. H. Terry, D. A. Christensen (2000), Vol. 3913, pp. 177-184, SPIE, San Jose, Calif.
12. Tolley, S., Wang, H., Smith, R., D A, C., and J N, H. (2003) Analytical Biochemistry 315, 223-237.
13. Pastinen, T., Raitio, M., Lindroos, K., Tainola, P., Peltonen, L., and Syvanen, A.-C. (2000) Genome Research 10, 1031-1042.
14. Herron, J. N., Christensen, D. A., Caldwell, K. D., Janatova, V., Huang, S.-C., and Wang, H.-K. (1993) in U.S. Pat. No. 5,512,492 to University of Utah Research Foundation.
15. Herron, J. N., Christensen, D. A., Caldwell, K. D., Wang, H.-K., Huang, S.-C., and Janatova, V., in U.S. Pat. No. 5,919,712 to University of Utah Research Foundation, U.S.
16. Herron, J. N., Christensen, D. A., Wang, H.-K., Caldwell, K. D., Janatova, V., and Huang, S.-C. (1997), in U.S. Pat. No. 5,677,196 to the University of Utah Research Foundation, U.S.
17. Kuppuswamy, M. N., Hoffmann, J. W., Kasper, C. K., Spitzer, S. G., Groce, S. L., and Bajaj, S. P. (1990) Proc. Natl. Acad. Sci. USA 88, 1143-1147.
18. Syvanen, A.-C., Aalto-Setala, K., Harju, L., Kontula, K., and Soderlund, H. (1990) Genomics 8,684-692.
19. (2002) Amersham Bioscience 27-2682-01.

1. A method of detecting a genetic polymorphism, the method comprising:
attaching a capture molecule to a solid support in at least one solution channel, wherein the at least one solution channel is in a flowcell and the solution channel is in close proximity with a waveguide;
injecting a sample, a polymerase, and a fluorescently labeled nucleotide into the at least one solution channel, under conditions wherein a single stranded analyte molecule present in the sample is capable of hybridizing to the capture molecule;
hybridizing the single stranded analyte molecule to the capture molecule;
extending the hybridized capture molecule by covalently adding a fluorescently labeled nucleotide to the hybridized capture molecule; and
detecting the presence or absence of the covalently added fluorescently labeled nucleotide.

2. The method according to claim 1, wherein the attaching a capture molecule to a solid support in at least one solution channel comprises attaching a plurality of different capture molecules patterned thereon to simultaneously detect different possible polymorphisms.

3. The method according to claim 1, wherein injecting the sample, the polymerase, and the nucleotide into the at least one solution channel comprises injecting at least two different fluorescently labeled nucleotides.

4. The method according to claim 3, wherein injecting the sample into the at least one solution channel comprises injecting the sample into at least two solution channels.

5. The method according to claim 1, wherein injecting the sample, the polymerase, and the nucleotide comprises injecting the polymerase and the sample prior to injecting the nucleotide.

6. The method according to claim 1, wherein the fluorescently labeled nucleotide comprises a Cy5-labeled dideoxynuclotide triphosphate.

7. The method according to claim 1, wherein the polymerase comprises thermostable DNA polymerase.

8. The method according to claim 7, wherein extending the hybridized capture molecule by covalently adding at least one fluorescently labeled nucleotide to the hybridized capture molecule is conducted at a temperature between about 40° C. and a about 50° C.

9. The method according to claim 8, wherein extending the hybridized capture molecule by covalently adding at least one fluorescently labeled nucleotide to the hybridized capture molecule is conducted at a pH between about pH 6 and about pH 8.5.

10. The method according to claim 1, wherein detecting the presence or absence of the covalently added fluorescently labeled nucleotide comprises detecting without washing the labeled nucleotide from the solution chamber.

11. The method according to claim 1, wherein extending the hybridized capture molecule by covalently adding at least one fluorescently labeled nucleotide to the hybridized capture molecule is conducted at a pH between about pH 6 and about pH 8.5.

12. The method according to claim 1, wherein detecting the presence or absence of the covalently added fluorescent nucleotide comprises using a CCD camera.

13. The method according to claim 12, wherein using the CCD camera comprises collecting data at 10-second intervals.

14. The method according to claim 13, wherein collecting data at 10-second intervals comprises collecting the data over a 5-minute period.

15. The method according to claim 14, wherein the flowcell comprises three solution channels, each in contact with approximately one-third of the waveguide.

16. The method according to claim 1, wherein injecting a sample, a polymerase, and a fluorescently labeled nucleotide into the at least one solution channel, under conditions wherein a single stranded analyte molecule present in the sample is capable of hybridizing to the capture molecule comprises injecting at least four labeled nucleotides, each nucleotide having a different fluorescent label, and utilizing multiplexing to simultaneously detect the addition of all four nucleotide possibilities in a single solution channel.

17. An assay system to detect a single nucleotide polymorphism, said assay system comprising:
a capture molecule attached to a solid support in contact with a solution channel, wherein the solution channel is in close proximity with a planar waveguide fluorescent biosensor;
a fluorescence biosensor capable of detecting a single base extension, wherein a single base extension comprises covalently attaching a fluorescently labeled nucleotide by the action of a polymerase to the capture molecule when a single stranded analyte molecule present in a sample is hybridized to the capture molecule.

18. The assay system of claim 17, comprising a wash-less assay system.

19. The assay system of claim 18, wherein the assay system utilizes multiplexing to simultaneously detect all base possibilities in a single solution channel.

20. An enzyme-catalyzed single base extension reaction used to detect single nucleotide polymorphisms with planar waveguide fluorescence biosensor technology.

21. A method of detecting genetic polymorphisms comprising using planar waveguides as the platform for single base extension.

22. An assay system to detect single nucleotide polymorphisms, said assay system comprising, in combination, a single base extension together with a planar waveguide fluorescent biosensor.

23. An enzyme-catalyzed single base extension reaction used to detect single nucleotide polymorphisms using planar waveguide fluorescence biosensor technology.

24. A wash-less assay comprising, in combination, a single base extension together with a planar waveguide.

25. The assay of any of the preceding claims wherein the waveguide platform has different capture molecules patterned thereon so as to simultaneously detect different possible polymorphisms.

26. The assay of any of the preceding claims wherein the SBEX assay utilizes multiplexing to simultaneously detect all base possibilities in a single channel.

27. An improved method of conducting a diagnosis using a waveguide, the improvement comprising: conducting a fluorescent affinity type assay, single base extension (SBEX), on said waveguide.


SEQUENCE LISTING (5 sequences)

SEQ ID NO: 1 - DNA, 21 bp, Artificial
  probe oligonucleotide
cctggcggaggatgaagacca

SEQ ID NO: 2 - DNA, 29 bp, Artificial
  Analyte G (Wild Type) captrue molecule
tccgtggtcttcatccaccgccaggagct

SEQ ID NO: 3 - DNA, 29 bp, Artificial
  Analyte A capture molecule
tccatggtcttcatccaccgccaggagct

SEQ ID NO: 4 - DNA, 29 bp, Artificial
  Analyte C capture molecule
tccctggtcttcatccaccgccaggagct

SEQ ID NO: 5 - DNA, 29 bp, Artificial
  Analyte T capture molecule
tccttggtcttcatccaccgccaggagct